(19) United States
(12) Patent Application Publication
(10) Pub. No.: US 2016/0188830 A1
(43) Pub. Date: Jun. 30, 2016

(54) SYSTEM AND METHOD FOR REAL-TIME ONLINE AND ON-DEMAND MEDICAL DIAGNOSIS AND TREATMENT OF A PATIENT

(71) Applicant: Mohamed M. Haq, Friendswood, TX (US)

(72) Inventor: Mohamed M. Haq, Friendswood, TX (US)

(21) Appl. No.: 14/985,193

(22) Filed: Dec. 30, 2015

Related U.S. Application Data
(60) Provisional application No. 62/124,739, filed on Dec. 31, 2014.

(51) Int. Cl. G06F 19/00; G06Q 50/14; A61B 7/02; A61B 1/227; A61B 8/00; G06Q 10/02; G06Q 50/22

(52) U.S. Cl. CPC G06F 19/3418; G06F 19/327; G06F 19/3431; G06Q 10/025; G06Q 50/14; G06Q 50/22; A61B 1/227; A61B 8/4444; A61B 7/02

(57) ABSTRACT

A system and method for enabling an on-demand real-time diagnosis and treatment of patients by remotely located physicians including a telemedicine clinic being configured to provide care to a patient; a monitor being configured to allow a physician to assess the patient from a remote location; a network being configured to enable the physician to communicate and diagnose the patient from the remote location; and a healthcare network website being configured to enable patients to make medical appointments.

### CROSS-REFERENCE TO RELATED APPLICATIONS

The present Utility patent application claims priority benefit of the [U.S. provisional application for patent Ser. No. 62/124,739 entitled “METHOD OF SELECTING A PERSONAL PHYSICIAN FOR A TELEMEDICINE CLINIC AND ITS LOCATION”, filed on 31 Dec. 2014 under 35 U.S.C. 119(e). The contents of this related provisional application are incorporated herein by reference for all purposes to the extent that such subject matter is not inconsistent herewith or limiting hereof.

### RELATED CO-PENDING U.S. PATENT APPLICATIONS

Not applicable.

### INCORPORATION BY REFERENCE OF SEQUENCE LISTING PROVIDED AS A TEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### FIELD OF THE INVENTION

One or more embodiments of the invention generally relate to on-demand real-time diagnosis and treatment of patients by qualified, remotely located physicians. More particularly, the invention relates to a method for selecting a primary care provider and location of a telemedicine clinic for facilitating a medical encounter between a patient at the telemedicine clinic and the primary care provider at a remote location from the patient.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The current medical diagnostic and treatment is generally performed at traditional medical clinics and establishments which may consist of physicians and other medical staff located in central office settings. The patient may visit the primary provider like an internist or family care physician at the central office location near their home or office for examinations, follow-up appointments, diagnostic tests, and treatment. If another specialist's opinions or treatments are required, the patient may have to schedule those appointments separately and subsequently visit those providers at a later date at other locations, sometimes far away. This may be a hardship for both patients and physicians. Patients, particularly for those patients located in rural areas or in countries having sparse medical coverage. Some relief may become available for homebound patients with telemedicine when they are able to receive home healthcare visits from a nurse in a mobile medical van, but this may create a hardship for the medical professional, who is unable to provide a volume of care to their patients due to travel time constraints, and cost still remains high.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that some patients may visit a Telemedicine clinic equipped with video conferencing capabilities and may interface with a remotely located physician having an association with the clinic in order to receive care. This arrangement is a small scale operation, and the patient may need access to the specific clinic having conferencing capabilities in order to interface with a physician. Although healthcare access may be provided in this manner, the patient is limited to receiving care from the physician(s) associated with the clinic. Geographic constraints may hinder access, quality and variety of healthcare for patients.

In situations where a patient is located away from their preferred doctor and possibly away from their insurance network, e.g., while traveling, locating and receiving affordable healthcare may be difficult. More people are traveling away from their home for business or vacation resulting in the inconvenience of locating a service that can provide healthcare and has access to their health records. If a patient is seen by a doctor outside of the patient's insurance network, for example, the patient may be responsible for all or at least a larger portion of the medical expenses incurred without getting similar level of service due to lack of records or history. As a result, patients may be constrained by their health insurance network as to where healthcare may be received.

Moreover, as medical specialties continue to evolve, the diagnosis and treatment methodology may involve many more providers and specialists than currently accessed. Healthcare networks are evolving or will evolve soon to allow patients to receive healthcare to places that are accessible by a communications network and proper equipment and personnel to convey the material data to a remote physician or multiple physicians so that the patient may receive high quality care regardless of their geographical location or the location of their health insurance company. Therefore, most of the routine medical care may be provided by a remote physician.

It is believed that what is needed is convenient location for a telemedicine clinic with desired equipment and network to access better suited primary care providers working with variety of specialists to provide high quality health care at lower cost.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a prior art Telemedicine clinic showing how the Patient and Nurse may video conference with the remote physician.

FIG. 2 illustrates a prior art Typical Neighborhood with housing, offices, schools, stores and medical clinics in any American city.

FIG. 3 illustrates an exemplary map of a state based on population density of each county, in accordance with an embodiment of the present invention.

FIG. 4 illustrates a prior art Telemedicine example with Primary care physician and multiple consulting physicians participating together in examining the patient.

FIG. 5 illustrates an exemplary Remote personal physician working with specialist to improve medical care flow diagram one cycle, in accordance with an embodiment of the present invention.

FIG. 6 illustrates an exemplary Remote personal physician working with specialist to improve medical care flow diagram two cycles, in accordance with an embodiment of the present invention.

FIG. 7 illustrates a prior art Airport clinic.

FIG. 8 illustrates an exemplary location to set up the exemplary telemedicine clinic at the Airport terminal, in accordance with an embodiment of the present invention.

FIG. 9 is a block diagram depicting an exemplary client/server system which may be used by an exemplary web-enabled/networked embodiment of the present invention.

FIG. 10 illustrates a block diagram depicting a conventional client/server communication system.

FIG. 11 illustrates a flow chart of an exemplary method for website activity based on flight status and delays adjusting an appointment time as necessary, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 9, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 99 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 99 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 99 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. §112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”, e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache . . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of” and “consisting essentially of” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

Telemedicine enables parties to engage in an on-demand communication session for the real-time diagnosis and treatment of patients by qualified, remotely located physicians. Communication sessions may be facilitated electronically by providing some or all participants with patient's medical records via a communications network including an operating console having at least a memory and at least a processor coupled to a healthcare network, the operating console configured for facilitating a medical encounter with complete video, audio and diagnostic equipments. The physician may have the ability to electronically prescribe medications for the patient using the same communications network.

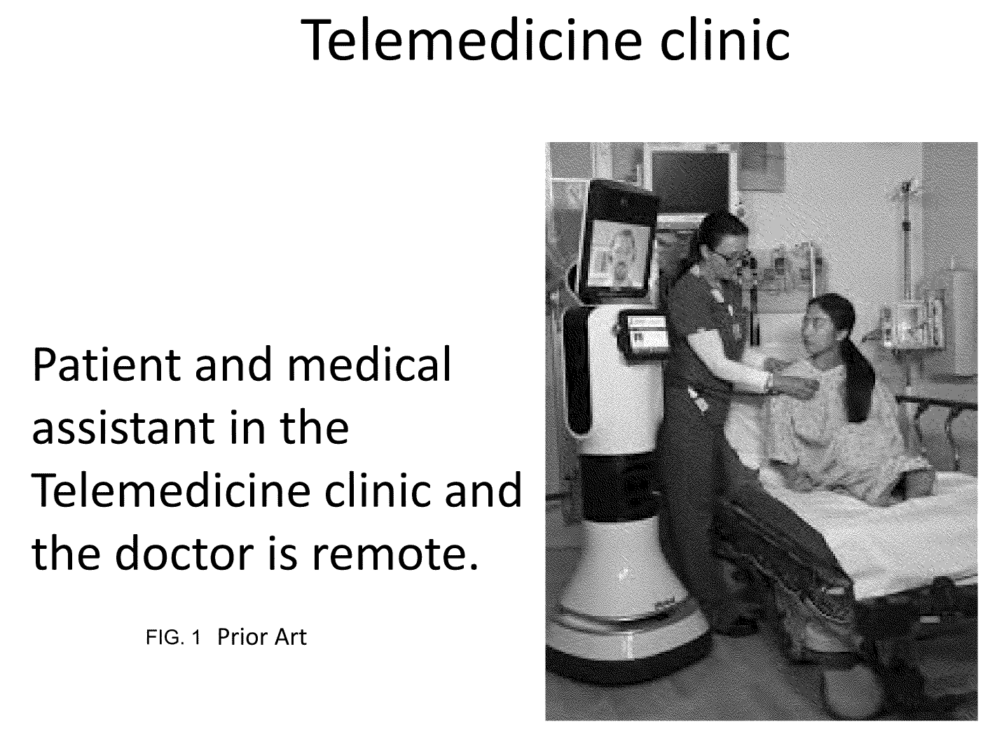

FIG. 1 depicts an exemplary telemedicine clinic where the patient is being helped by a medical assistant with a wireless stethoscope to send the signals to a remote physician shown in the monitor. The Telemedicine clinic also has other diagnostic and measurement tools like General Examination Camera which has 1-50× zoom capability that allows close focus as well a wide angle video, Digital Otoscope and USB Ultrasound Probes. This allows the physicians to assess the patient entirely from a remote location. In one embodiment, the telemedicine clinic may have specialized equipment like, but not limited to, a Digital ENT Otoscope, a Dermascope, a 12 Lead ECG, a Digital Spirometer, USB Ultrasound Probes, an Audiometer and Various Endoscopes as needed by the various specialists. The physician is decoupled from the patient and may be located anywhere data may be transmitted through a network. This allows the physician to be at home or even a golf course and attend to a patient during his/her round of golf his gives the patient method to receive medical care from his physician using a telemedicine clinic located at major airport around the world.

Selecting Remote Personal Physician.

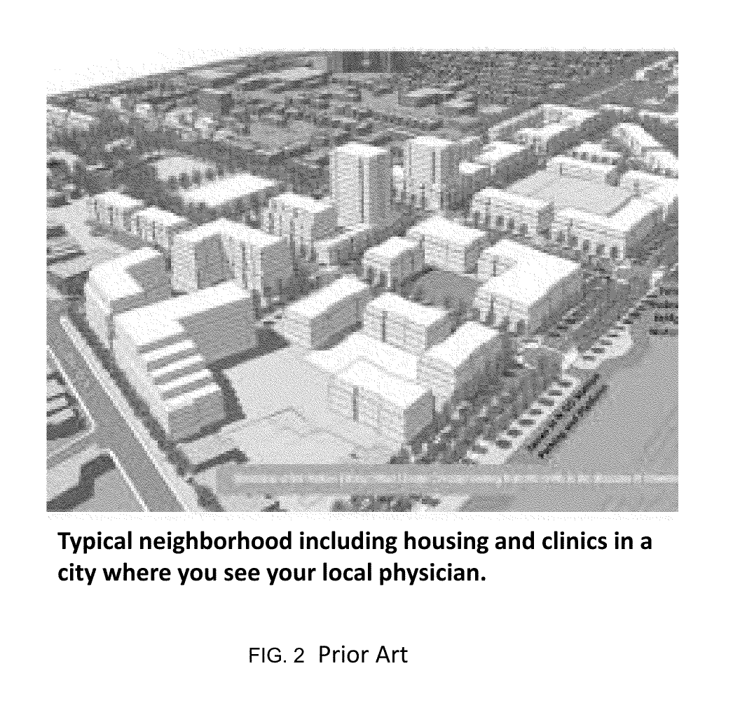

FIG. 2 shows a prior art typical city neighborhood which has housing, schools, offices, stores and medical clinic in close vicinity. This is typically where a patient may have a personal physician from a small group of available physicians. If a Remote Personal Physician is chosen, a patient may have a much wider choice of physicians from the whole licensing area. The patient may visit the primary care provider like an internist at the central office location near their home or office for examinations, follow-up appointments, diagnostic tests, and treatment.

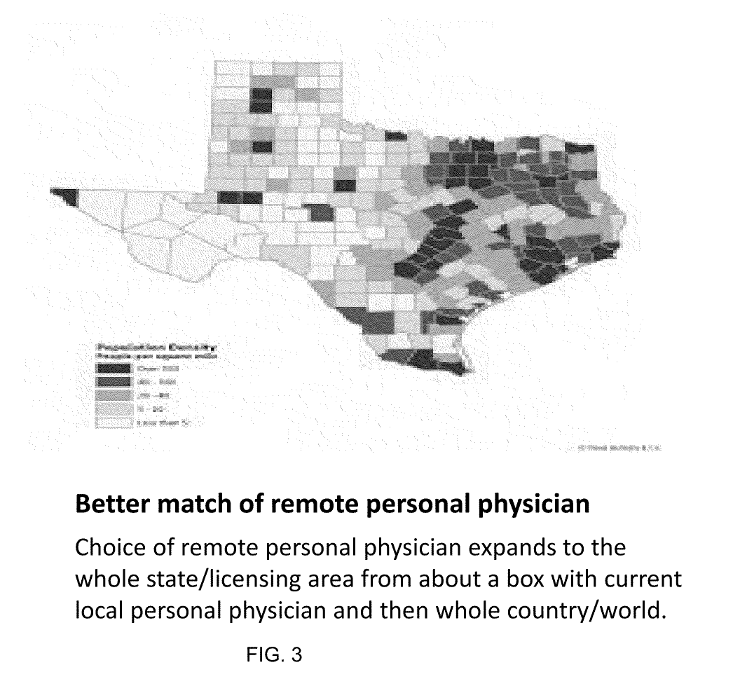

FIG. 3 shows an exemplary state and its population density. There may be more than twenty-five thousand primary care physicians in the whole state. Almost all the physicians may be capable of being online and may connect to a healthcare network. Most of physicians may be available at one or more online clinics connected to healthcare networks if they desire. This allows the telemedicine patients to get a better match with a physician based on desired gender, age, ethnicity, language, lifestyle, interest and alma mater. If the physician and the patient have similar age and interest like running or bicycling they may easily discuss topics like diet, hydration or injuries related to those activities. Similarly, if they are the same ethnicity and speak the same language they understand their diet or customs making it easier to convey the symptoms or issues more easily than a physician of different ethnicity. The choice for patients from rural areas may go up by 1000× when most of the physicians are available online. Even for the urban area patients the choice goes up by 50-100×. The better matched primary care physician may improve the quality of care in general compared to the few available in the neighborhood.

A typical American after finishing high school relocates more than nine times in life. The relocation may be due to multiple factors like job, marital status, and addition to the family, cost or change in the demographics of the neighborhood etc. It is easier to keep the same Remote Personal Physician if the licensing and insurance requirements are met, as compared to a neighborhood physician. It is similar to land line number being related to neighborhood or zip code and the cell number is transportable even beyond the state boundaries. It is more than likely that law would change in the near future allowing Remote Personal Physician to remain the same even if the relocation is beyond the state boundary.

The remote personal physician who is in demand may choose his patients based on profession, zip code, education, alma mater, age, gender, language or ethnicity. The physician may prefer patients who are educated, well to do and successful because they tend to follow instructions and have a healthier life style in general. Some physicians may want athletes as he/she is big sports fan, or they may prefer patients who have been to certain schools, etc. Better matched physicians and patients may improve the overall quality of medical care. A remote personal physician could be an existing primary care physician which is seen in the neighborhood office.

Some foreigners who visits the US often may prefer a remote personal physician in the US that speaks their language and are of the same ethnic background. Some foreigners may prefer a remote personal physician for consultation in the US based on skills and reputation of physicians in US, as compared to their local physician. If they have access to a telemedicine clinic in their local area, which may connect them to reputable physicians in US, they may choose to be treated by US physicians if permitted by applicable laws in the respective countries. American expats living in various countries may choose their remote personal physicians in the US through a telemedicine clinic. Similarly, Japanese expats working in various countries including the US, for example, working in the Automotive or electronics industry may see their remote personal physician in Japan. The telemedicine clinics contemplates various combinations of patients located in a country where they are living, but having remote personal physician in another country of their choice based on physician's skill, expertise, language or ethnic background etc. The same patients may also see the same remote personal physicians while travelling anywhere in the world from a telemedicine clinic available in the vicinity.

Improve Methodology for Personal Physician Working with Specialists.

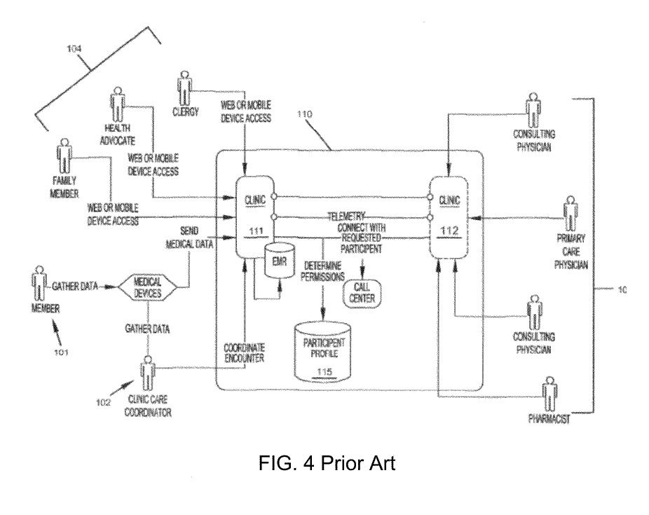

Medical care methodology needs to evolve to care for complex cases where multiple physicians are needed for diagnosis and treatment of patients. FIG. 4. illustrates a prior art where multiple consulting physicians and the primary care physician are remotely examining a patient. This arrangement for a complex case with significant symptoms and signs is fine. But for routine checkups without symptoms this is expensive and an inconvenient method. The insurance pays only after authorization for consulting physicians (specialists) with the appropriate symptoms. Generally, the primary care physician is not aware of every possible risk factor for patients of certain ethnic or racial background. Waiting for symptoms to occur may make the medical issue more expensive or difficult to treat. If the patient is of Asian Indian background and is a non-smoker, normal weight and does not eat meat (the normal risk factors for Heart disease) the primary care physician may not be worried about heart disease for the patient, if they are not also of Asian Indian background.

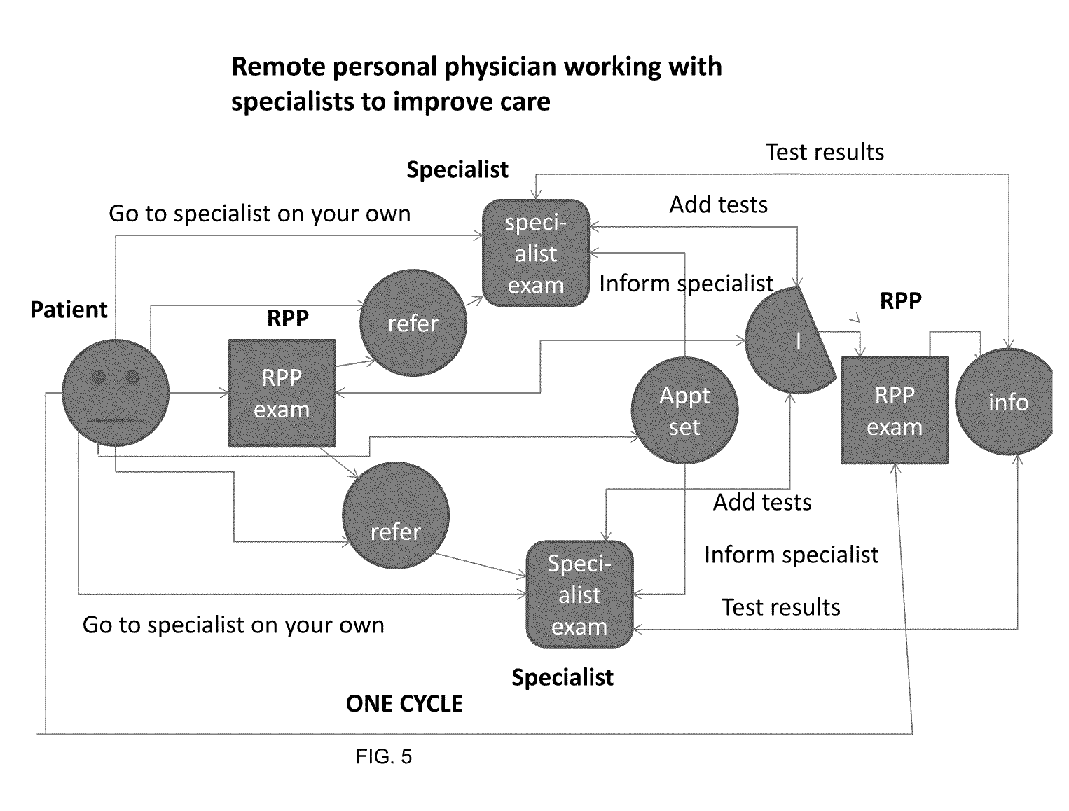

FIG. 5 shows an exemplary methodology for improving care from a primary physician working with specialists, in accordance with an embodiment of the present invention. If a cardiologist is involved, the cardiologist would suggest “LIPOPROTEIN a” test for risk stratification, especially applicable to this patient population. Normally an internist OR primary care physician do not check for HDL3, they just check HDL, LDL, Cholesterol and triglycerides. But middle aged Caucasians may have additional risk factor for coronary heart disease later in life which maybe be detected earlier by checking the HDL3. A cardiologist would likely add this test based on family history even if the other factors and symptoms don't show any risk. Caucasian women of child bearing age increase their risk of heart attack or stroke by thirteen fold if they smoke and take birth control pills. If a cardiologist is involved they may suggest alternate birth control method to reduce the risk.

Similarly, if a patient lab tests shows less hemoglobin and fewer red blood cells in their body than normal during a regular checkup and patient has no symptoms, the primary care physician may look at it as anemia. The primary care physician may prescribe Iron supplements or add iron rich foods like meat to the patient diet as treatment. A Hematologist looking at the same lab data and the racial/ethnic profile of the patient as African American or Mediterranean origin may consider the possibility of Thalassemia. The Hematologist may conduct additional tests to determine whether it is mild Thalassemia which does not need any treatment and actually worsens with excess iron. The hematologist may tell the patient to avoid infections, eat a healthy balanced diet and get vaccinated against for pneumococcal, flu and hepatitis B.

The patient may have high blood pressure (hypertension) for years without any symptoms. It damages the blood vessels and heart increasing the risk for serious health problems like heart attack or stroke. Fortunately, high blood pressure may be easily detected, but the treatment of hypertension is different for African American, than a person who has diabetes or a person who has kidney problem. If the primary care physician works with a specialist like endocrinologist or nephrologists than the treatment protocol may be better suited with fewer side effects. Similarly, a patient with common variable immune deficiency (CVID) may go often to the primary care physician with recurrent infections, which is progressively harder to treat with antibiotics, but a specialist in Infectious diseases may diagnose it as CVID quickly. Then the treatment may be immunoglobulin replacement therapy (IRT) rather than giving lot of antibiotics every time there is an infection.

A normal personal care physician or family practice physician examines about 4000-6000 patients a year, more than 90 percent of the patients come for routine checkup or for cold, cough, fever or bacterial infection etc. However, they have to evaluate the entire spectrum of diseases involving the body. The likelihood that the primary care physician may detect the early onset of a serious disease is very low, particularly if only a handful (OR 3-5 out of 5000) of their patients have marginal lab results, abnormal examination findings, combination of risk factors or are of a different ethnic background.

FIG. 5 shows an exemplary method to improve patient care by involving specialists (experts or consulting physicians) with a remote personal physician, in accordance with an embodiment of the present invention. In Step S1, the patient has a checkup from a telemedicine clinic with a remote personal physician (RPP) for the first time. A routine physical, blood tests, etc is performed to establish a medical record. Based on the profile and the examination, the RPP may refer the patient to see one or two more specialists if needed, in Step S2a. Also it may be helpful in getting improved medical profile based on family history, lifestyle, local or occupational risk factors related to the patients by seeing specialists, in Step 3a. Step S3a is performed by a patient when a patient goes to the specialist for a consultation on their own. Treatments required from the examination by the RPP and the specialists may be based on the findings and available medical record at that time. But if abnormal findings are found on the first visit with the RPP, like chest pains, higher blood pressure or abnormal lab findings, the related specialist is consulted before prescribing any medications. The patient profile and medical record is updated with data from the RPP and two specialist physicians like a hematologist and cardiologist, for example, as discussed earlier in this section. In another embodiment, if two or more specialists are involved, the specialist related to the abnormal finding may continue to have encounter in the second cycle while the specialist related to normal finding is only informed of results and may not require an encounter in the next cycle. This completes one cycle (yearly cycle) before the patient sees the RPP again. In an alternative embodiment, it is not necessary that the patient has to see only two specialists in the first cycle. The patient may see one specialist or multiple specialists, the method remain the same as long as one specialist has examined remotely or in person. In another embodiment, the patient may see the RPP either in person or remotely for any symptoms more often or as needed, and appropriately related specialist (if the patient already had an encounter with that specialist) is alerted for his input prior to the examination of the Patient. In additional embodiments, the specialist may be involved with evaluation of lab results and medication if they are needed. A specialist sees about 1000-2000 patients annually. Most of the cases fall within a narrow spectrum of diseases. The chances of catching or suspecting abnormal test results or examination findings by a specialist are at least five to ten times higher than a personal physician. The specialist may inform the patient for a follow up test immediately or in the near future depending on the severity of abnormality to verify or rule out the suspicion, in Step S4.

When the patient sets up an appointment (Step S4) with an RPP for the next yearly checkup (Steps S1, S2b, S3b), all the specialists which had an encounter with the patient are informed. In Step S5, the specialists add simple tests which are incorporated in the RPP examination (Step S6), routine blood test or urine/stool test etc based on the medical records and their previous encounter. After the examination by the RPP, lab tests are shared by all the involved physicians, in Step S7, shown as “info” in a circle. The method of the present embodiment may be automated to inform all the involved physicians when the next appointment is made and after results of tests and examination are received.

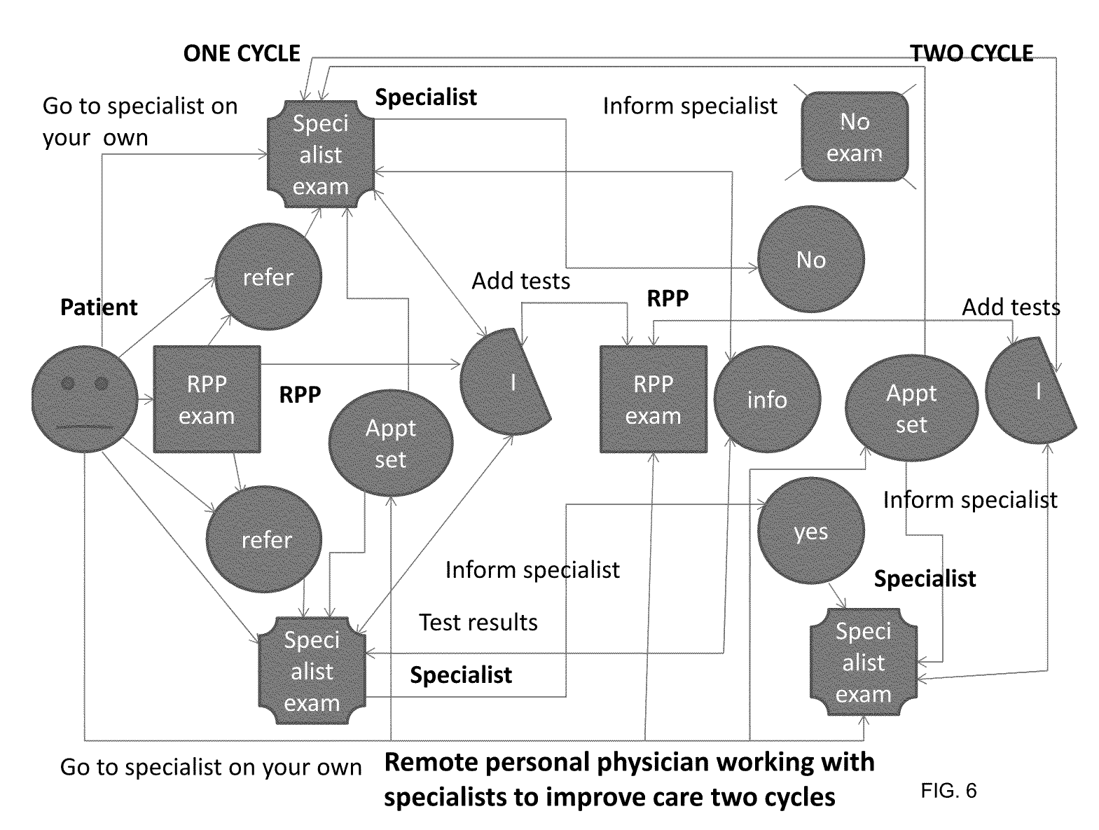

FIG. 6 shows an exemplary method to improve patient care by involving specialists (experts or consulting physicians) with a remote personal physician, in accordance with an embodiment of the present invention. In Step S1, the patient has a checkup from a telemedicine clinic with a remote personal physician (RPP) for the first time. A routine physical, blood tests, etc is performed to establish a medical record. Based on the profile and the examination, the RPP may refer the patient to see one or two more specialists if needed, in Step S2a. Also it may be helpful in getting improved medical profile based on family history, lifestyle, local or occupational risk factors related to the patients by seeing specialists, in Step 3a. Step S3a is performed by a patient when a patient goes to the specialist for a consultation on their own. Treatments required from the examination by the RPP and the specialists may be based on the findings and available medical record at that time. But if abnormal findings are found on the first visit with the RPP, like chest pains, higher blood pressure or abnormal lab findings, the related specialist is consulted before prescribing any medications. The patient profile and medical record is updated with data from the RPP and two specialist physicians like a hematologist and cardiologist, for example, as discussed earlier in this section. This completes one cycle (yearly cycle) before the patient sees the RPP again. It is not necessary that the patient has to see only two specialists in the first cycle. The patient may see one specialist or multiple specialists, the method remains the same as long as one specialist has examined remotely or in person. The patient may see the RPP either in person or remotely for any symptoms more often or as needed, and appropriately related specialist (if the patient already had an encounter with that specialist) is alerted for his input prior to the examination of the Patient. The specialist may be involved with evaluation of lab results and medication if they are needed. A specialist sees about 1000-2000 patients annually. Most of the cases fall within a narrow spectrum of diseases. The chances of catching or suspecting abnormal test results or examination findings by a specialist are at least five to ten times higher than a personal physician. The specialist may inform the patient for a follow up test immediately or in the near future depending on the severity of abnormality to verify or rule out the suspicion, in Step S4.

When the patient sets up an appointment (Step S4) with an RPP for the next yearly checkup (Steps S1, S2b, S3b), all the specialists which had an encounter with the patient are informed. In Step S5, the specialists add simple tests which are incorporated in the RPP examination (Step S6), routine blood test or urine/stool test etc based on the medical records and their previous encounter. After the examination by the RPP, lab tests are shared by all the involved physicians, in Step S7. If the results are within the expected range from the previous encounter, the respective specialist physician does not need to see the patient (Step S8) unless the symptoms warrant it, until the next RPP exam in the next cycle, shown as the crossed out specialist exam. If an unexpected result occurs during examination or lab tests the related specialist request the patient for a follow up in Step S9. This is shown as “yes” in a circle which leads to specialist examining the patient again in Step S10. If there is any treatment, all the related specialists are informed.

Annual blood testing and other diagnostic lab testing is the most important step aging adults may take to prevent life-threatening disease. The blood test results may allow a specialist to catch critical changes in the body before they manifest as heart disease, cancer, diabetes, etc. The benefits of blood test go far beyond disease prevention; blood tests may monitor levels of sex hormones, so a patient may take decisive steps to enhance their quality of life. The blood tests may be used to correct a depressive mental state, erectile dysfunction, abdominal obesity, or by improving the patient memory and energy levels.

As years go by more and more specialists may be added allowing the patient to be monitored by 5-10 specialists every year without having to see most of them during the year. The specialists are involved in defining the diagnostic tests, interpreting the result and recommending the medications jointly with the RPP. This method of multiple specialists monitoring the patient may improve the chances of catching serious diseases before their onset by an order of magnitude compared to patient only seeing the personal physician until the onset of symptoms. This improves the level of care for patient and allows them to adjust their lifestyle early before the risk factors increase. The RPP may improve care with help from specialists and patient to avoid getting treated by medications which may aggravate some other condition. If the patient starts adding specialists while he/she is healthy, say between 30-50 years of age it may add decades of healthy life to the person.

Telemedicine Clinic Location.

The location of a healthcare facility needs to be convenient and accessible for lots of people. If it is a location people come for other reasons, then it saves them an extra trip, when they go to see the physician. That would be more efficient. If a patient have to spend some time in the healthcare facility, whether planned or unplanned, then it would be a more efficient use of the patient time to complete routine medical checkups or follow ups with their remote personal physician and/or other remote specialists at a telemedicine clinic location.

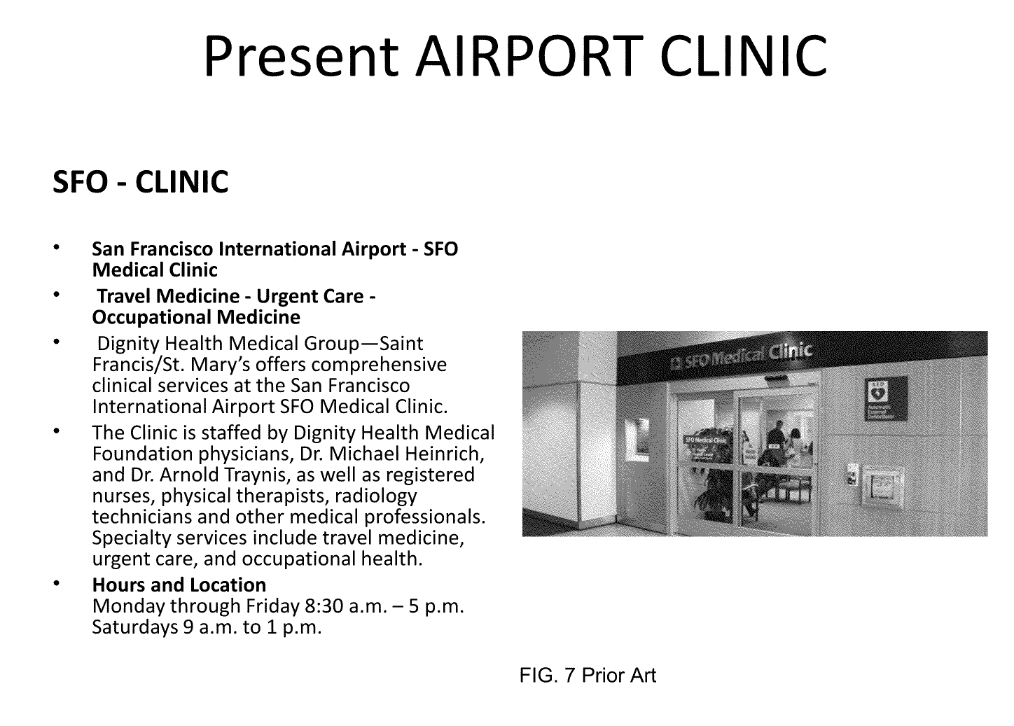

FIG. 7 shows an exemplary Airport clinic, in accordance with an embodiment of the present invention. It is set up for Urgent care, Travel Medicine or Occupational Medicine. People travelling should be healthy; otherwise they may have to postpone their travel. The clinic is open from 8.30 am to 5.00 pm five days a week and half day Saturday. About thirty to fifty thousand passengers may fly from a major Airport on the average every day. When adding the people who work at the airport, people who come to pick up or drop passengers etc, then the number of people becomes 15 to 25% higher. The top 10 major airports in the U.S has about 30,000-100,000 passengers on average every day.

The passengers who are flying may have to reach the airport ahead of their scheduled time of departure due to uncertainty from traffic, security lines, and baggage check-in if needed. Most of the people who are flying from small airports may have to change planes in the hub airports to reach their destination. The time between check-in and departure or during the layover could be utilized for appointment with a physician if available through a telemedicine clinic located at the major airports. At a destination airport, there may be 15-20 minutes wait for baggage to arrive. There may be an extra wait time on top of this to rent a car or wait for a ride. It may be convenient to come earlier than a departure time to complete a scheduled appointment with the physician at the telemedicine clinic. Similarly, the appointment may be set a little after the scheduled landing time. The time people spend at the airport increases, if the passengers are travelling overseas. So every passenger who is travelling has four opportunities during a round trip to schedule an appointment with a remote personal physician if the Telemedicine clinic is available at those Airports. First, when the person leaves the local city airport and second when the person lands at the destination airport. On the way back the person follows at least the same two airports. If there is a change of planes to reach the destination, the person has more opportunities to utilize the time at the airport to see the remote personal physician online. The airport telemedicine clinic may operate on the same hours as the airport itself, since the remote personal physician may see patients from their homes also. If a patient travel route is frequent from airports and the major airports have a telemedicine clinic connected to a healthcare network having the remote personal physicians available, then it is easy to set up appointments a little ahead of the takeoff or little later after landing to see a desired remote personal physician.

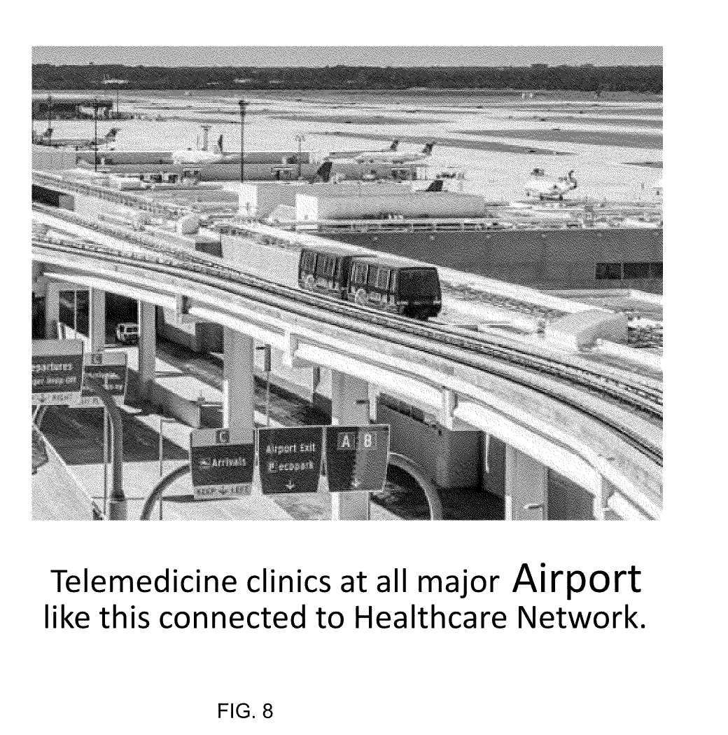

FIG. 8 illustrates an exemplary location to set up the exemplary telemedicine clinic at the Airport terminal, in accordance with an embodiment of the present invention. The telemedicine clinic location shown has the needed diagnostic equipment, and is contemplated to be an improvement to the prior art telemedicine clinic shown in FIG. 1 connected to a healthcare network at major airports around the world, in accordance with various embodiment of the present invention. By having telemedicine clinics connected to a healthcare network operating at most large airports in the U.S and around the world, it would be much more efficient for patients to schedule appointments and see their remote personal physicians at an airport telemedicine clinic. It may save patients a trip of going to a neighborhood physician's office. It may also save airport telemedicine clinic patients time if the airport telemedicine clinic patients utilize layover at hub airports, baggage arrival time or time between check-in and departure and also allow them to see a remote physician if possible, when there are unscheduled delays. A personal physician can be seen as a Remote Personal Physician from an Airport telemedicine clinic overseas, if it is convenient, even if both live in the same neighborhood. By having telemedicine clinics in dozens of airports in the U.S and around the world working together, a large portion of the population may efficiently see their remote personal physician for routine medical care and/or follow up or specialists for consultation etc. It may be convenient to expand to additional airports worldwide once the demand for telemedicine clinics increases. It is also possible to add telemedicine clinics in multiple terminals of a busy airport for convenience. In addition, a patient doesn't have to worry about searching new personal physicians when relocating. By selecting a better matched Remote Personal Physician, avoiding the drive to a neighborhood personal physician, utilizing the time available at the airport to complete the routine checkup, follow up and seeing specialists when needed may improve the quality of medical care. Airport telemedicine clinics may be more convenient for people who live in rural areas and travel by plane at least few times a year. Rural areas often have few physicians and putting a telemedicine clinic in rural areas may be expensive and inconvenient for nurses and staff if they get few patients.

In alternative embodiments, Foreigners may use the airport telemedicine clinic in their country to consult with a remote personal physician in the US. They may have their own remote personal physician and have their routine checkups or follow-ups etc if the law permits. If desired, foreign patients may have multiple physicians including their own remote personal physician in the US, a licensed local physician in their country, both of whom may jointly examine the patient, following up with needed blood or lab tests, and treatment including medication, if necessary. As discussed in the Selection of remote personal physician section, a Japanese working in the US may see his remote personal physician from the airport close to a work location if a telemedicine clinic is available. Travelers around the world may access their remote personal physician from any airport telemedicine clinic, making it convenient for people to access their medical records and see the physicians they are familiar with easily.

Subscription Model or Membership Model.

Prior to selecting a Remote Personal Physician, most people may have their neighborhood personal physician. So the current insurance provider may not pay for getting another physician Similarly the insurance provider may not cover for encounters with a specialist without symptoms, authorization and reference from the neighborhood personal physicians.

In an embodiment, the business model contemplates a subscription based model. People may enroll or become members of a healthcare network operating telemedicine clinics at major airports around the world for a yearly fee which covers a routine yearly checkup with a matched RPP and one encounter with a specialist, as shown in FIGS. 5 and/or 6. Other services or appointments are charged at the prevailing rate with appropriate discounts for the subscribers. Over time one may drop a neighborhood physician or may need Insurance coverage, if desired. The subscription fee may also pays a specialist involvement with the RPP prior to a yearly checkup, including, but not limited to, lab test interpretations and medications.

In additional embodiments, the annual membership fee may be like insurance in foreign countries for travelers. Foreign travelers who come to the U.S for visits may establish an RPP. Then foreign travelers may be able consult a physician, if needed. The membership may be set up to see a local licensed physician in case of emergency from every airport in the world where the telemedicine clinic operates at a discounted price. For example, if a patient wants to see an RPP in the U.S, while in a Tokyo Airport at an airport telemedicine clinic, arrangement is made with a Japanese physician to complete the prescription online based on consultation with the patient and the RPP, if it is permitted by local laws. This is very convenient since there is no need to locate a local physician who may speak the same language or has the patient medical record. This subscription works for any traveler who becomes a member and selects a remote personal physician during travels to another foreign country where the airport telemedicine clinic is available. Foreigners who prefer personal physicians in the US may also become members and consult with them for routine checkup or follow-up remotely. This may improve the level of service worldwide and people may prefer the best doctors available online to service them regardless of geographic location, if the applicable local law allows it. Since telemedicine clinics are located in multiple airports worldwide, at least some of may be open for any patient to access their medical record for emergency purpose. Having access to medical records may be a useful alert for patient's family also. If a subscriber is involved in an accident or requires emergency treatment the required authorities may use the subscribers identification to obtain the subscribers medical record and the airport telemedicine clinic may alert the family.

In alternative embodiments, the various processes described earlier like selection of Remote Personal Physician, Improved methodology for Personal Physician to work with specialist or the healthcare network telemedicine clinics operating at major airports worldwide or the subscription model may operate on its own or in combination with one or two other processes also. A patient may see a Remote personal physician from a local pharmacy if a telemedicine clinic is available there, but a lot of neighborhood pharmacies have to operate the telemedicine clinics in most of the cities around the world to be useful for large segment of the population making investment much larger than the investment for about 30-50 airports worldwide. The improved methodology for personal physician to work with a specialist is useful by itself or in combination with remote personal physician, but the current insurance does not pay without the specialist examining the patient and/or without any symptoms. The current insurance only pays for physical checkup with a personal physician not for specialist without symptoms. The airport telemedicine clinic may work by itself if a patient sees any available physician, but may not be useful in improving long-term quality of medical care as the physician may not be familiar with the patient, similar to an urgent care clinic.

In other additional embodiments, the combination of Remote Personal Physician, Airport telemedicine clinic connected to a healthcare network, and improved methodology for remote physician working closely with specialists may improve the quality of medical care and add decades of healthy and vibrant life to the consumers. It may save a trip to a physician's office and a referral to a specialist after the onset of symptoms and thus utilizing time efficiently at any airport where a telemedicine clinic is available. The better matched Personal Physician using improved methodology reduces the chances of missing abnormal risk which are associated with race, ethnicity, heredity in combination of habits, by an order of magnitude. The remote personal physician may provide basic medical coverage for travelers in foreign countries with the airport clinic for consultation of simple cold, cough, sore throat, body pains etc. The remote personal physician has the patient's medical record including allergies and other conditions which might be useful for prescribing medicines directly or through a local licensed physician. The patient's medical records are available in case of emergency in a foreign country, since some of the telemedicine clinics worldwide are open and may access the records immediately. The patient doesn't have to change the remote personal physician or the airport telemedicine clinic if the patient relocates since medical care is conducted online. The subscription model is the quickest way to achieve the desired results.

Although the present invention has been described with reference to few preferred embodiments, especially the lab tests for determining the diagnosis, or interpreting the lab results and interaction of medications with other existing conditions, persons skilled in the art may recognize that changes may be made in form and detail without departing from the spirit and scope of the invention.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that may be carried out on a computing machine, a typical computer system may, when appropriately configured or designed, serve as a computer system in which those aspects of the invention may be embodied.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that may be carried out on a computing machine, a typical computer system may, when appropriately configured or designed, serve as a computer system in which those aspects of the invention may be embodied. Thus, the present invention is not limited to any particular tangible means of implementation.

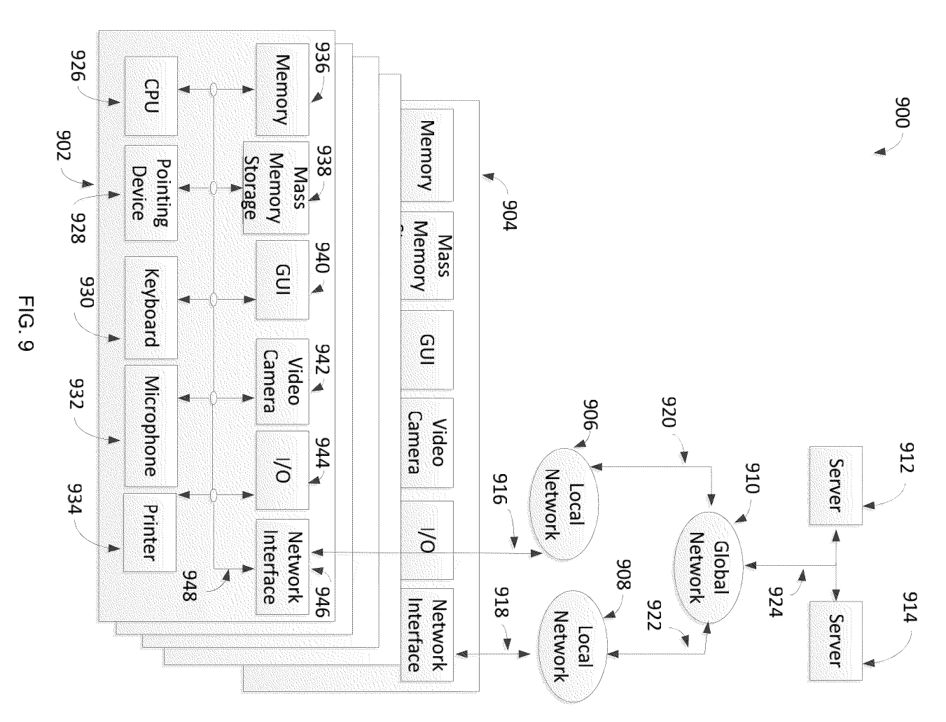

FIG. 9 is a block diagram depicting an exemplary client/server system which may be used by the exemplary web-enabled/networked telemedicine clinic embodiment of the present invention.

A communication system 900 includes a multiplicity of clients including, but not limited to, a telemedicine clinic client device, a remote physician client device, a healthcare network website, etc., with a sampling of clients denoted as a client 902 and a client 904, a multiplicity of local networks with a sampling of networks denoted as a local network 906 and a local network 908, a global network 910 and a multiplicity of servers with a sampling of servers denoted as a server 912 and a server 914.

Client 902 may communicate bi-directionally with local network 906 via a communication channel 916. Client 904 may communicate bi-directionally with local network 908 via a communication channel 918. Local network 906 may communicate bi-directionally with global network 910 via a communication channel 920. Local network 908 may communicate bi-directionally with global network 910 via a communication channel 922. Global network 910 may communicate bi-directionally with server 912 and server 914 via a communication channel 924. Server 912 and server 914 may communicate bi-directionally with each other via communication channel 924. Furthermore, clients 902, 904, local networks 906, 908, global network 910 and servers 912, 914 may each communicate bi-directionally with each other.

In one embodiment, global network 910 may operate as the Internet. It will be understood by those skilled in the art that communication system 900 may take many different forms. Non-limiting examples of forms for communication system 900 include local area networks (LANs), wide area networks (WANs), wired telephone networks, wireless networks, or any other network supporting data communication between respective entities.

Clients 902 and 904 may take many different forms. Non-limiting examples of clients 902 and 904 include personal computers, personal digital assistants (PDAs), cellular phones and smartphones.

Client 902 includes a CPU 926, a pointing device 928, a keyboard 930, a microphone 932, a printer 934, a memory 936, a mass memory storage 938, a GUI 940, a video camera 942, an input/output interface 944 and a network interface 946.

CPU 926, pointing device 928, keyboard 930, microphone 932, printer 934, memory 936, mass memory storage 938, GUI 940, video camera 942, input/output interface 944 and network interface 946 may communicate in a unidirectional manner or a bi-directional manner with each other via a communication channel 948. Communication channel 948 may be configured as a single communication channel or a multiplicity of communication channels.

CPU 926 may be comprised of a single processor or multiple processors. CPU 926 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 936 is used typically to transfer data and instructions to CPU 926 in a bi-directional manner. Memory 936, as discussed previously, may include any suitable computer-readable media, intended for data storage, such as those described above excluding any wired or wireless transmissions unless specifically noted. Mass memory storage 938 may also be coupled bi-directionally to CPU 926 and provides additional data storage capacity and may include any of the computer-readable media described above. Mass memory storage 938 may be used to store programs, data and the like and is typically a secondary storage medium such as a hard disk. It will be appreciated that the information retained within mass memory storage 938, may, in appropriate cases, be incorporated in standard fashion as part of memory 936 as virtual memory.

CPU 926 may be coupled to GUI 940. GUI 940 enables a user to view the operation of computer operating system and software. CPU 926 may be coupled to pointing device 928. Non-limiting examples of pointing device 928 include computer mouse, trackball and touchpad. Pointing device 928 enables a user with the capability to maneuver a computer cursor about the viewing area of GUI 940 and select areas or features in the viewing area of GUI 940. CPU 926 may be coupled to keyboard 930. Keyboard 930 enables a user with the capability to input alphanumeric textual information to CPU 926. CPU 926 may be coupled to microphone 932. Microphone 932 enables audio produced by a user to be recorded, processed and communicated by CPU 926. CPU 926 may be connected to printer 934. Printer 934 enables a user with the capability to print information to a sheet of paper. CPU 926 may be connected to video camera 942. Video camera 942 enables video produced or captured by user to be recorded, processed and communicated by CPU 926.

CPU 926 may also be coupled to input/output interface 944 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 926 optionally may be coupled to network interface 946 which enables communication with an external device such as a database or a remote computer or telecommunications or internet network using an external connection shown generally as communication channel 916, which may be implemented as a hardwired or wireless communications link using suitable conventional technologies. With such a connection, CPU 926 might receive information from the network, or might output information to a network in the course of performing the method steps described in the teachings of the present invention, but not limited to, remote communication and diagnosis of a patient at the telemedicine clinic with a physician located remotely from the telemedicine clinic.

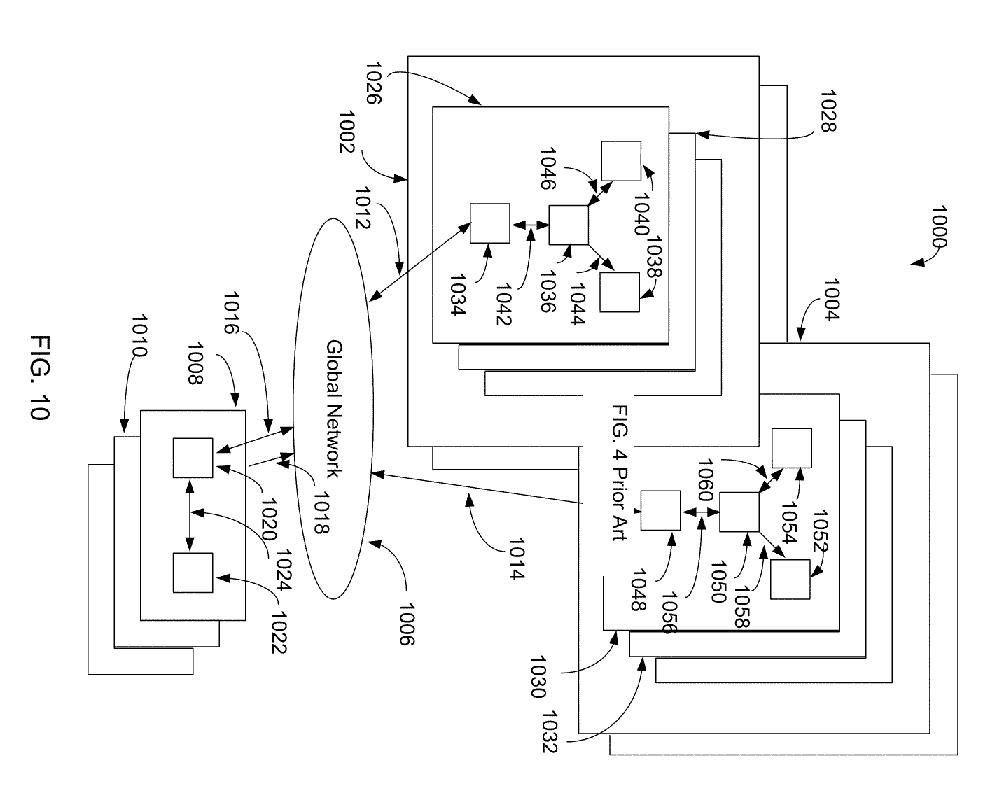

FIG. 10 illustrates a block diagram depicting a conventional client/server communication system.

A communication system 1000 includes a multiplicity of networked regions with a sampling of regions denoted as a network region 1002 and a network region 1004, a global network 1006 and a multiplicity of servers with a sampling of servers denoted as a server device 1008 and a server device 1010.

Network region 1002 and network region 1004 may operate to represent a network contained within a geographical area or region. Non-limiting examples of representations for the geographical areas for the networked regions may include postal zip codes, telephone area codes, states, counties, cities and countries. Elements within network region 1002 and 1004 may operate to communicate with external elements within other networked regions or within elements contained within the same network region.

In some implementations, global network 1006 may operate as the Internet. It will be understood by those skilled in the art that communication system 1000 may take many different forms. Non-limiting examples of forms for communication system 1000 include local area networks (LANs), wide area networks (WANs), wired telephone networks, cellular telephone networks or any other network supporting data communication between respective entities via hardwired or wireless communication networks. Global network 1006 may operate to transfer information between the various networked elements.

Server device 1008 and server device 1010 may operate to execute software instructions, store information, support database operations and communicate with other networked elements. Non-limiting examples of software and scripting languages which may be executed on server device 1008 and server device 1010 include C, C++, C# and Java.

Network region 1002 may operate to communicate bi-directionally with global network 1006 via a communication channel 1012. Network region 1004 may operate to communicate bi-directionally with global network 1006 via a communication channel 1014. Server device 1008 may operate to communicate bi-directionally with global network 1006 via a communication channel 1016. Server device 1010 may operate to communicate bi-directionally with global network 1006 via a communication channel 1018. Network region 1002 and 1004, global network 1006 and server devices 1008 and 1010 may operate to communicate with each other and with every other networked device located within communication system 1000.

Server device 1008 includes a networking device 1020 and a server 1022. Networking device 1020 may operate to communicate bi-directionally with global network 1006 via communication channel 1016 and with server 1022 via a communication channel 1024. Server 1022 may operate to execute software instructions and store information.

Network region 1002 includes a multiplicity of clients with a sampling denoted as a client 1026 and a client 1028. Client 1026 includes a networking device 1034, a processor 1036, a GUI 1038 and an interface device 1040. Non-limiting examples of devices for GUI 1038 include monitors, televisions, cellular telephones, smartphones and PDAs (Personal Digital Assistants). Non-limiting examples of interface device 1040 include pointing device, mouse, trackball, scanner and printer. Networking device 1034 may communicate bi-directionally with global network 1006 via communication channel 1012 and with processor 1036 via a communication channel 1042. GUI 1038 may receive information from processor 1036 via a communication channel 1044 for presentation to a user for viewing. Interface device 1040 may operate to send control information to processor 1036 and to receive information from processor 1036 via a communication channel 1046. Network region 1004 includes a multiplicity of clients with a sampling denoted as a client 1030 and a client 1032. Client 1030 includes a networking device 1048, a processor 1050, a GUI 1052 and an interface device 1054. Non-limiting examples of devices for GUI 1038 include monitors, televisions, cellular telephones, smartphones and PDAs (Personal Digital Assistants). Non-limiting examples of interface device 1040 include pointing devices, mousse, trackballs, scanners and printers. Networking device 1048 may communicate bi-directionally with global network 1006 via communication channel 1014 and with processor 1050 via a communication channel 1056. GUI 1052 may receive information from processor 1050 via a communication channel 1058 for presentation to a user for viewing. Interface device 1054 may operate to send control information to processor 1050 and to receive information from processor 1050 via a communication channel 1060.

For example, consider the case where a user interfacing with client 1026 may want to execute a networked application. A user may enter the IP (Internet Protocol) address for the networked application using interface device 1040. The IP address information may be communicated to processor 1036 via communication channel 1046. Processor 1036 may then communicate the IP address information to networking device 1034 via communication channel 1042. Networking device 1034 may then communicate the IP address information to global network 1006 via communication channel 1012. Global network 1006 may then communicate the IP address information to networking device 1020 of server device 1008 via communication channel 1016. Networking device 1020 may then communicate the IP address information to server 1022 via communication channel 1024. Server 1022 may receive the IP address information and after processing the IP address information may communicate return information to networking device 1020 via communication channel 1024. Networking device 1020 may communicate the return information to global network 1006 via communication channel 1016. Global network 1006 may communicate the return information to networking device 1034 via communication channel 1012. Networking device 1034 may communicate the return information to processor 1036 via communication channel 1042. Processor 10106 may communicate the return information to GUI 10108 via communication channel 1044. User may then view the return information on GUI 1038.

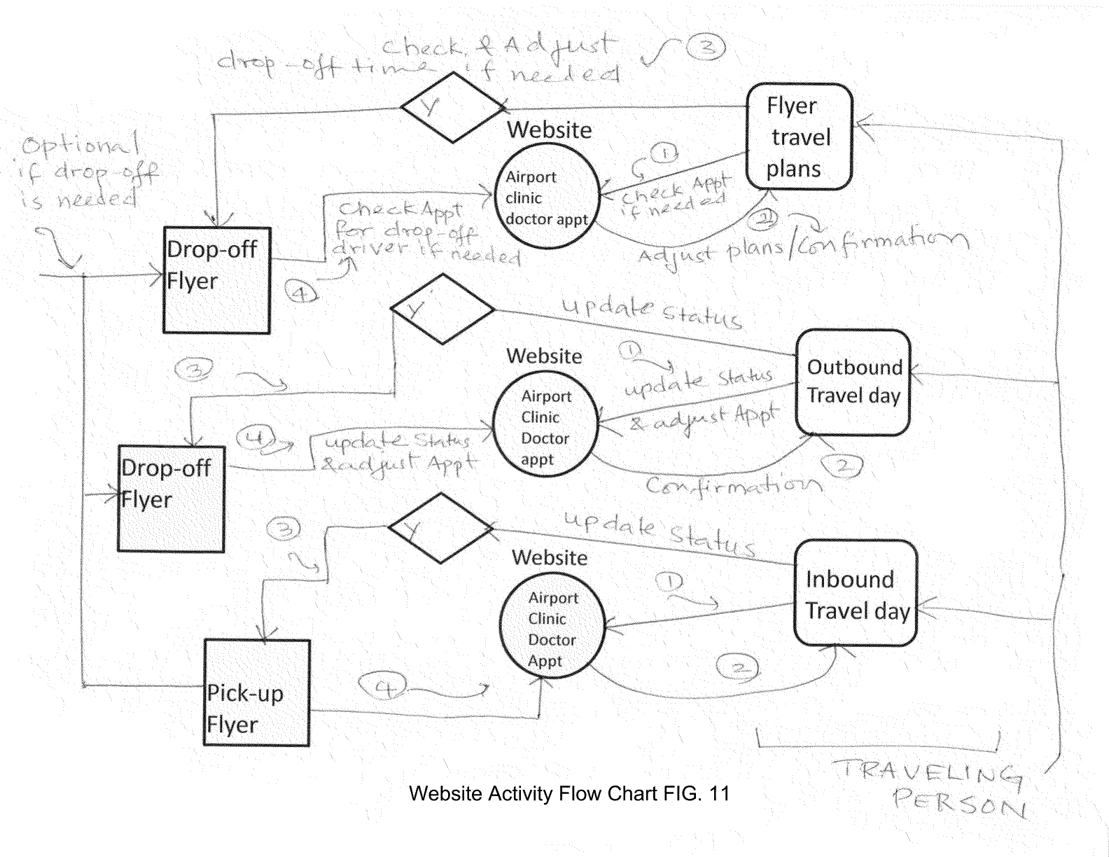

FIG. 11 illustrates a flow chart of an exemplary method for website activity based on flight status and delays adjusting an appointment time as necessary, in accordance with an embodiment of the present invention.

In the present embodiment, patient medical care appointments are done online through the healthcare network website as shown in FIG. 11. The website may be used to set up appointments, update status of flights which has patients travelling or planning to travel on them. When the member planning to travel initiates his travel plans as shown on top right hand side of the FIG. 11, it also looks for the appointments if needed as shown in step (1), if needed he adjust his flight schedule or appointment to best suit his travel plans is shown in step (2). If the member requires a drop-off, then the step (3) is used to set up and adjust the drop-off time. The person dropping off the traveler can also set up an appointment at the airport clinic as shown in step in step (4). The appointments contain the flight information for the patients if they flying. The airport telemedicine clinic has real time flight information of the related flights on display, so the scheduled patient may easily see the updated schedule for their flight. This information may also be available on the website. The real time flight schedule may be updated to inform the physician of the likelihood of the patient's appointment needs to be adjusted on the day of travel as shown as Outbound Travel Day. The same steps (1) thru (4) can be checked and updated as needed. If the physician adjusts the appointment window, then the patient may be informed by text and also on the website. This is repeated also for the inbound travel if they appointments for that segment. As more and more people become members of the healthcare network to use the airport telemedicine clinic, the website activity increases at a much higher rate as appointments start to fill-up at peak times. Patients have to access the website multiple times to find the most convenient schedule or adjust the schedule if the best slots are already reserved. They have to go back to the website and see if reserved slots have opened up later to find best possible time slot. The patients may visit the travel reservation website followed by an airport clinic website or the other way around depending upon the available choices. The patients may check the airline flight status and their appointment window if the flight is off schedule. The website may do the billing online. The website may have interviews with medical experts, patient reviews, health and nutrition related articles. It may also update the availability of expert physicians to answer questions from patients. The website activity allows additional revenue from targeted advertisement based on health and travel related products and services. The advertisement may be based on whether the website visitors are business travelers or for leisure. The advertisement may also be based on information whether the patient is returning home or staying for a short duration in a hotel etc. This may be accomplished without compromising the privacy issues of the patients.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC §112 (6) it is intended that the corresponding means for and/or steps for carrying out the claimed function are the ones that are locally implemented within the jurisdiction of the USA, while the remaining aspect(s) performed or located remotely outside the USA are not intended to be construed under 35 USC §112 (6). In some embodiments, the methods and/or system components which may be located and/or performed remotely include, without limitation:

It is noted that according to USA law, all claims must be set forth as a coherent, cooperating set of limitations that work in functional combination to achieve a useful result as a whole. Accordingly, for any claim having functional limitations interpreted under 35 USC §112 (6) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC §112 (6) is/(are) dependent on one or more remote servers located outside the USA, it is intended that each such recited function under 35 USC §112 (6) is to be interpreted as the function of the local system receiving the remotely generated information required by a locally implemented claim limitation, wherein the structures and or steps which enable, and breathe life into the expression of such functions claimed under 35 USC §112 (6) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present application, and “35 USC §112 (6)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing an on-demand real-time diagnosis and treatment of patients by qualified, remotely located physicians according to the present invention will be apparent to those skilled in the art. Various aspects of the invention have been described above by way of illustration, and the specific embodiments disclosed are not intended to limit the invention to the particular forms disclosed. The particular implementation of the method for on-demand real-time diagnosis and treatment of patients by qualified, remotely located physicians may vary depending upon the particular context or application. By way of example, and not limitation, the on-demand real-time diagnosis and treatment of patients by qualified, remotely located physicians described in the foregoing were principally directed to a system and method for selecting a primary care provider and location of a telemedicine clinic for facilitating a medical encounter between a patient at the telemedicine clinic and a provider at a remote location from the patient implementations; however, similar techniques may instead be applied to mobile networked systems for remote care of patients, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A system comprising:
means for providing care to a patient;
means for examining a patient at the care providing means, by a physician located remotely from the care providing means, said patient examining means comprises means for communicating with the patient and diagnosing the patient from the remote location.

2. The system of claim 1, further comprising means for selecting and making an appointment with a Remote Personal Physician at the patient care providing means.

3. The system of claim 1, further comprising means for subscribing patients at the patient care providing means, wherein said patient care providing means comprises a plurality of patient care providing means located at various locations.

4. The system of claim 1, in which said patient care providing means comprises a plurality of patient care providing means located at major airports being configured to provide patient health care for travelling patients.

5. The system of claim 4, further comprising a means for allowing the patient to make an online appointment at the airport patient care providing means.

6. The system of claim 5, in which said online appointment comprises a flight information of the patient.

7. A system comprising:
a telemedicine clinic being configured to provide care to a patient;
a monitor being configured to enable a physician to assess the patient from a remote location, said monitor comprises;
a camera being configured to provide a close focus and wide angle video viewing;
a display being configured to be operable for visual communication with the patient;
a digital otoscope;
a USB ultrasound probe;
a network being configured to enable the physician to communicate and diagnose the patient from the remote location;
a healthcare network website being configured to enable the patient to make appointments to the telemedicine clinic.

8. The system of claim 7, further comprising a stethoscope being configured to send heart rate signals to the remote physician being shown in the display.

9. The system of claim 7, further comprising a Digital ENT Otoscope.

10. The system of claim 7, further comprising a Dermascope.

11. The system of claim 7, further comprising a 12 Lead ECG.

12. The system of claim 7, further comprising a Digital Spirometer.

13. The system of claim 7, further comprising an Audiometer.

14. The system of claim 7, further comprising an Endoscope.

15. The system of claim 7, wherein said telemedicine clinic comprises a plurality of telemedicine clinics located at various airport locations being configured to provide health care to travelling patients.

16. A method comprising the steps for:
selecting, with a healthcare network website, a personal physician who is licensed to practice in a state of a patient residence;
selecting, with the healthcare network website, a remote personal physician in the state for routine medical needs using a telemedicine clinic;
communicating and diagnosing the patient at the telemedicine clinic from the remote location;
calculating future risk factors based on ethnic background or race to improve a health care of the patient.

17. The method if claim 16, further comprising the step for making an online appointment at the telemedicine clinic with the healthcare network website, wherein the telemedicine clinic is located at an airport location, and in which the appointment comprises a flight information of the patient.

18. The method of claim 17, further comprising the step for updating the flight information of the patient to inform the physician if a scheduled appointment time window needs to be adjusted.

19. The method of claim 18, further comprising the step for alerting the patient of an adjusted appointment time window.

20. The method of claim 19, further comprising the step for updating the availability of expert physicians to answer questions from the patient.
